(19) United States
(12) Patent Application Publication
(10) Pub. No.: US 2012/0108960 A1
(43) Pub. Date: May 3, 2012

(54) METHOD AND SYSTEM FOR ORGANIZING STORED ULTRASOUND DATA

(76) Inventors: Menachem (Nahi) Halmann, Bayside, WI (US); Mark Steven Urness, Wauwatosa, WI (US)

(21) Appl. No.: 12/939,047

(22) Filed: Nov. 3, 2010

(51) Int. Cl. A61B 8/00; G06F 3/048

(52) U.S. Cl. 600/437; 715/810; 715/839; 715/823; 715/781

(57) ABSTRACT

Methods and systems for organizing stored ultrasound data are provided. One method includes displaying selectable anatomical identification guidance information having a plurality of identifiers corresponding to a plurality of anatomical portions of an anatomical region and receiving a user input selecting one of the plurality of identifiers. The method further includes storing a subsequently acquired image and associating the stored image with the anatomical potion of the anatomical region corresponding to the selected identifier.

### BACKGROUND OF THE INVENTION

The subject matter disclosed herein relates generally to methods and systems for organizing stored ultrasound data, and more particularly to organizing ultrasound data stored over time to be subsequently displayed.

Diagnostic medical imaging systems typically include a scan portion and a control portion having a display. For example, ultrasound imaging systems usually include ultrasound scanning devices, such as ultrasound probes having transducers that are connected to an ultrasound system to control the acquisition of ultrasound data by performing various ultrasound scans (e.g., imaging a volume or body). The ultrasound systems are controllable to operate in different modes of operation and to perform different scans. The acquired ultrasound data then may be displayed, which may include images of a region of interest.

When using ultrasound for anatomical based procedures, such as scanning the same portion of a patient (e.g., a finger) over time to determine the effectiveness of a treatment, the conventional review process is very time consuming. For example, using conventional ultrasound systems, it is very time consuming for doctors to perform comparative image analysis on the ultrasound system to review an image from a past exam while performing a live exam. In particular, the doctor may have to search through a large number of stored images to review the image from the past exam, thereafter remember the area he or she is looking at, and then switch back to the live exam or view a live image on another display. This process is not only time consuming, but can lead to errors, such as by selecting the wrong past image or when switching between image views.

Moreover, when performing ultrasound scanning, the scanning parameters are unique to each piece or portion of anatomy being scanned. Using conventional ultrasound systems, the user has to remember or manually modify each parameter for the anatomy being scanned. This is also a time consuming process and can lead to the potential for error when different scan parameters are used between exams.

Thus, when performing long term treatment assessment using ultrasound data, the review process, as well as the process for performing subsequent scans for comparison can be time consuming and prone to errors in review.

### BRIEF DESCRIPTION OF THE INVENTION

In accordance with various embodiments, a method for storing ultrasound data is provided. The method includes displaying selectable anatomical identification guidance information having a plurality of identifiers corresponding to a plurality of anatomical portions of an anatomical region and receiving a user input selecting one of the plurality of identifiers. The method further includes storing a subsequently acquired image and associating the stored image with the anatomical potion of the anatomical region corresponding to the selected identifier.

In accordance with other various embodiments, an ultrasound display is provided that includes a first section displaying a currently acquired ultrasound image of an anatomical portion of a patient and a second section displaying an archived ultrasound image of the anatomical portion of the patient. The ultrasound display further includes a third section displaying selectable anatomical identification guidance information having a highlighted identifier corresponding to the anatomical portion.

In accordance with yet other various embodiments, an ultrasound system is provided that includes a probe configured to acquire ultrasound image data and a memory storing the acquired ultrasound image data using a predefined anatomical identification arrangement. The ultrasound system further includes a processor configured to obtain archived ultrasound image data and a display configured to display an image based on the acquired image data and an image based on the archived image data stored in the memory. The display further displays selectable anatomical identification guidance information having a highlighted identifier corresponding to an anatomical portion for the displayed images.

### BRIEF DESCRIPTION OF THE DRAWINGS

FIG. 1 is a block diagram illustrating a process for organizing archived ultrasound data in accordance with various embodiments.

FIG. 2 is a diagram illustrating an exemplary database organization structure for storing ultrasound data accordance with various embodiments.

FIG. 3 is a screenshot of an exemplary display in accordance with an embodiment.

FIG. 4 is a screenshot of an exemplary display in accordance with another embodiment.

FIG. 5 is a flowchart of a method to acquire and store ultrasound data in accordance with various embodiments.

FIG. 6 is a block diagram of an ultrasound system in which various embodiments may be implemented.

FIG. 7 is a block diagram of an ultrasound processor module of the ultrasound system of FIG. 6 formed in accordance with various embodiments.

FIG. 8 is a diagram illustrating a three-dimensional (3D) capable miniaturized ultrasound system in which various embodiments may be implemented.

FIG. 9 is a diagram illustrating a 3D capable hand carried or pocket-sized ultrasound imaging system in which various embodiments may be implemented.

FIG. 10 is a diagram illustrating a 3D capable console type ultrasound imaging system in which various embodiments may be implemented.

### DETAILED DESCRIPTION OF THE INVENTION

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide a system and method for organizing stored ultrasound data, such as archived ultrasound data from multiple exams over time. For example, the stored ultrasound data may be acquired from multiple exams for a particular anatomical region to assess the images, such as to compare images of the same anatomical regions over time. At least one technical effect of various embodiments is easier access and display of archived ultrasound images from previous ultrasound exams. By practicing various embodiments, the evaluation of historical ultrasound images for comparison may be provided to determine, for example, treatment progress.

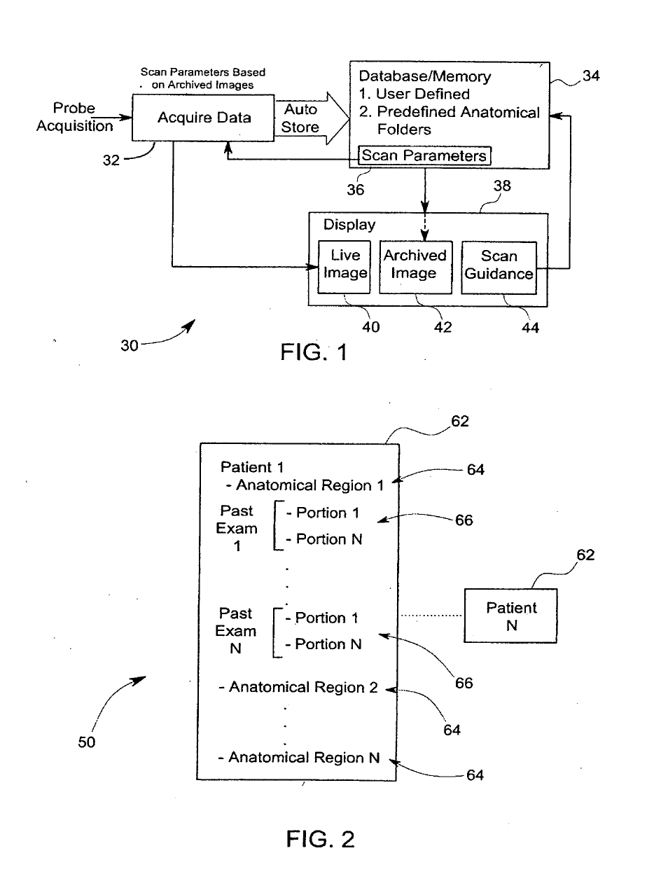

One embodiment of a process 30 for organizing archived ultrasound data and controlling the acquisition of ultrasound data for a plurality of exams over time is illustrated in FIG. 1. The process 30 includes acquiring ultrasound data at 32, which may include image data for a particular anatomical region of interest, such as one or more fingers of a patient. For example, an ultrasound probe is used to acquire image data with the scan parameters optionally set or adjusted based on scan parameters corresponding to previously acquired ultrasound images for the same anatomical region. Thus, the scan parameters for a current ultrasound scan may be based on user defined settings, predefined settings for the particular exam, or based on scan parameters corresponding to archived images and to which the currently acquired images are to be compared.

As the ultrasound image data is acquired, the ultrasound image data is stored, and more particularly, automatically stored at 34 in a database or memory. The image data is stored automatically for the particular patient being imaged such that the image data is subsequently accessible for that patient. Additionally, the image data is automatically organized in the database or memory. In various embodiments, the automatic storage of the image data may include storing the image data based on user defined settings and/or in predefined anatomical folders corresponding to the anatomical region being imaged. The archived image data is thereafter accessible based on a selected anatomical region or area such that in various embodiments only archived ultrasound images for a selected anatomical region or area are accessible and presented to a user for display. The archived image data may be stored in a hierarchical manner in memory with the particular memory locations associated with the anatomical regions or areas.

It should be noted that the scan parameters, for example, user defined settings for scan parameters 36 corresponding to the archived images also may be stored and associated with each of the images. Accordingly, the scan parameters for subsequent scans of the same anatomical region or area may be selected or automatically set based on the stored scan parameters 36. It should be noted that other information may be stored in connection with or associated with the stored ultrasound image data. For example, in some embodiments a definition of the image processing algorithm used to process the image data is also stored with the stored images. Accordingly, if the system software is, for example, upgraded, then a follow-up scan may be performed using the same algorithms (and parameters) as in the previous exam. The algorithms may be any suitable types of algorithms including, for example, Power Doppler processing, Speckle Reduction, etc.

The process 30 also includes displaying image data at 38, such as on a display having multiple display portions or sections. Thus, a single display may present to a user multiple pieces of information for a particular anatomical region or area. For example, in various embodiments, a first section 40 of the display screen may display a live image or real-time image received from the ultrasound probe and corresponding to a currently acquired image, such as an image of an anatomical region or area being imaged as part of a current ultrasound exam. A second section 42 of the display screen may display an archived image from a prior exam corresponding to the same anatomical region or area as the live image. For example, the images in the first and second sections 40 and 42 may be displayed in a side by side manner in a simultaneous or concurrent manner. A third section 44 of the display screen may display scan guidance information corresponding to the anatomical region or area being imaged and selectable by a user. For example, the third section 44 may include an anatomical image legend highlighting the anatomical region or area corresponding to the displayed archived image. In other embodiments, the third section 44 may include a customized user-defined table, which includes text, such as a hierarchical tree structure, that describes and corresponds to the archived ultrasound images to facilitate scanning the same area. This correlation of the information in the third section 44 with the images displayed in the first and section sections 40 and 42 facilitates displaying appropriate images (e.g., images from the same anatomical region) for comparison.

In operation, as the user navigates through the legend or user-defined table, such as by making different selections, the archived ultrasound image(s) corresponding to the selection is made accessible and/or displayed automatically. Additionally, during this navigation process, which can define the different image views to be acquired for an exam or a portion thereof, the scan parameters or imaging presets of the ultrasound device acquiring the image(s) may be automatically set or adjusted to the settings used from a prior exam, for example, corresponding to the archived image being displayed. Thus, the scanning parameters of the ultrasound device in some embodiments are automatically changed based on each archived image that is selected, which can allow a user (e.g., a doctor) to more easily and/or quickly access optimized images for each portion of anatomy being scanned.

In various embodiments, a database organization structure 60, such as an organization structure for automatically organizing archived ultrasound data in a patient and anatomical associated manner is illustrated in FIG. 2. The structure 60, which may be stored in any suitable manner and using any suitable storage technique, includes storing image data for each patient in a corresponding patient record 62 such that all images for a particular patient are accessible by selecting a particular patient record. For example, a user may enter a patient's name or identification number in the ultrasound system to access the patient record 62. It should be noted that although specific details for only one patient record 62 is shown, additional patient records for the same or different patients may be provided in a similar manner.

The structure 60 is organized in an anatomical based arrangement, which may be provided, for example, as a hierarchical anatomical based storage arrangement. In some embodiments, for each patient record 62, a plurality of anatomically defined and associated memory locations is provided for each patient record 62. For example, anatomical region memory locations 64 are provided that identify memory for storing images for a particular anatomical region or area, such as a hand or finger for each of a plurality of past exams. Each of the anatomical region memory locations 64 may further be defined or divided based on anatomical portions, such as corresponding to images of joints of a finger, to thereby define anatomical portion memory locations 66.

The archived ultrasound image data, and any associated or accompanying data, may be stored either locally (e.g., stored within the ultrasound system) or remotely, such as over a wired, wireless and/or interne network, among others. Thus, the storage size is not limited to the storage capabilities of the ultrasound system. Additionally, with remote storage and subsequent access of the archived data, subsequent follow-up exams may be performed, for example, on another ultrasound system (that includes similar capabilities as described herein).

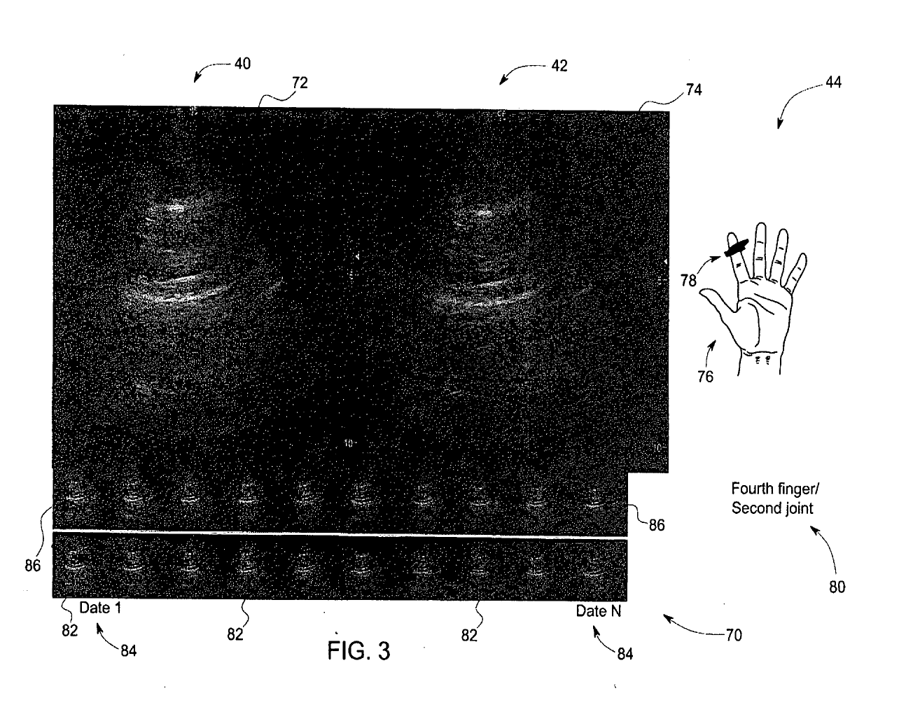

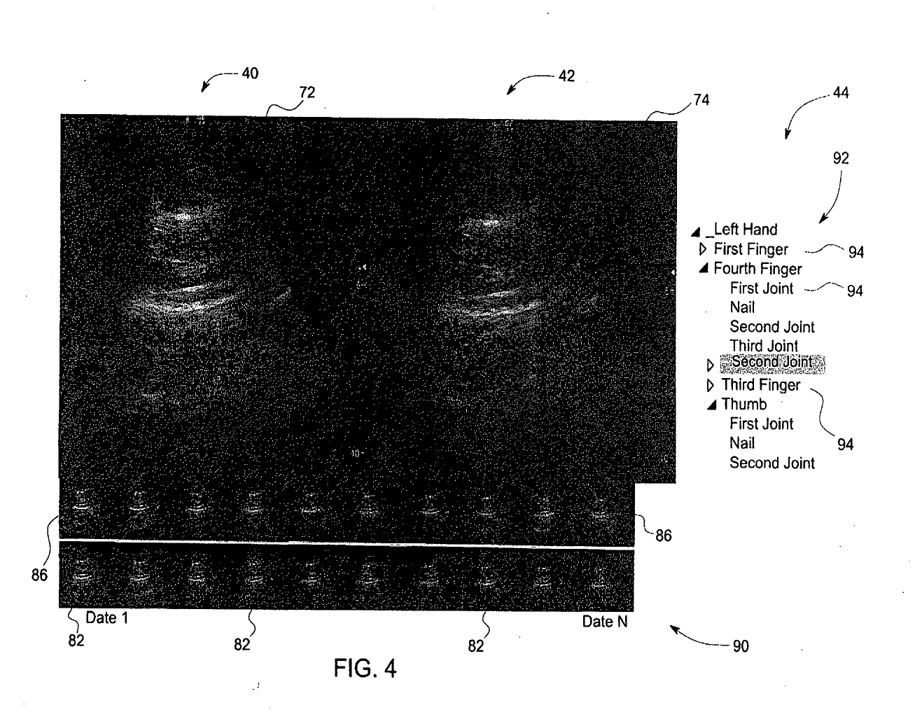

Various embodiments provide for organizing archived data that is accessible based on user selection with a user interface, such as illustrated in FIGS. 3 and 4, showing exemplary displays of an ultrasound system. For example, a wide screen display of an ultrasound display may include different sections as described in more detail herein. With the display, a user can use the anatomical legend or customize a table to define a scan sequence and provide scan guidance for a user. In some embodiments, the scan guidance can be a customized study, which may be based on a particular practice or department. Additionally, ultrasound user per image scan parameters are provided without having to manually document or remember the parameters.

With the display 70 of FIG. 3 or the display 90 of FIG. 4, selectable anatomical information (e.g., scan guidance information) allows for loading the correct anatomical reference image (e.g., archived image), re-calibrating all scan parameters based on the archived image and resuming scanning with a live image displayed. Such selectable operation may be provided with single selection functionality (e.g., one click operation), by selecting the anatomical region or area of interest to be scanned next.

More particularly, FIG. 3 illustrates the display 70 with the first section 40, second section 42 and third section 44. In the display 70, the first and second sections 40 and 42 define image display regions, which in some embodiments display a live and archived image, respectively, of the same anatomical region. However, it should be noted that variations and modifications are contemplated. For example, the order of the displayed images may be changed or two archived images may be displayed instead.

In the illustrated embodiment, the first section 40 displays a live ultrasound image 72 and the second section 42 displays an archived image 74. The images 72 and 74 correspond to an imaged anatomical region identified and/or selected in the third section 44. For example, the third section 44 in this embodiment includes an anatomical image legend 76, which in this embodiment is a hand. However, it should be noted that any anatomical region or area may be represented by the anatomical image legend 76. For example, a plurality of preloaded and/or predefined anatomical image legends 76 may be provided based on the images to be acquired and displayed. In other embodiments, a user may create and/or modify the anatomical image legend 76, for example, based on the particular exam, particular patient characteristics, etc.

The anatomical image legend 76 is selectable, such as with a pointer and user input device (e.g., mouse). For example, in some embodiments a plurality of selectable portions 78 (one is shown highlighted in the FIG. 3) of the anatomical image legend 76 may be defined, such that a user can select any one of the portions 78. Accordingly, in these embodiments, user selectable “hot spots” on which a user may click or select are provided. However, in other embodiments, any portion of the anatomical image legend 76 may be selected by a user. The portions 78 may correspond, for example, to those portions of the anatomical region that are scanned as part of an ultrasound exam. When the portion 78 is selected by a user, that portion 78 is identified on the display, for example, by highlighting (e.g., yellow highlighting) that portion 78, which is illustrated as a joint of a finger of the hand in the anatomical image legend 76. However, any type of highlighting may be provided. Additionally, a text description 80 of the selected portion 78 may be provided, such as a text description of the selected joint. For example, in the illustrated embodiment, the text description 80 defines or labels the selected finger and joint. This descriptor may be assigned to all images stored in memory corresponding to this portion 78 of the anatomical image legend 76 and for which the anatomy is scanned.

When a user selects a portion 78, the user also identifies the next scan portion of the anatomical region patient that is going to be acquired (or identifying a portion corresponding to archived images of interest). Upon selecting the portion 78, archived images 82 for that portion 78 are displayed, such as thumbnail images. For example, the archived images 82 may correspond to images from some or all of the previous scans for the particular finger and joint. A user may scroll through the archived images 82 and not all of which may be displayed on the screen. Each displayed archived image 82 also may include a date identifier 84 corresponding to the date on which each of the archived images 82 were acquired. Thus, the archived images 82 for the selected portion 78 are automatically accessed and displayed, which are then selectable by a user. Upon selection of one of the displayed archived images 82 that image is displayed in the second section 42, for example, as the enlarged image 74. Upon acquisition of a current image of the portion 78, that image is displayed in the first section 40, for example, as the enlarged image 72. It should be noted that live images 86 for all of the portions 78 previously scanned during the current exam also may be displayed, for example, as thumbnail images.

It should be noted that in various embodiments, when displaying images from multiple exams, some post-processing parameters may be applied to all of the displayed images, which may be performed simultaneously or currently to all of the displayed images. For example, a Gain or Look Up Table selection affects all displayed images (and not just the active image) such that all of the images maintain the same scan setup. Accordingly, this post-processing may be performed on all displayed images.

As another example, and as illustrated in FIG. 4, the display 90 includes a table 92, which is illustrated as a list arranged as a hierarchical tree that includes text descriptors 94 of the various portions of the anatomical region for the particular exam. Similar to the anatomical image legend 76, the table 92 identifies the portions of the anatomical region to be scanned with a user selected text descriptor 94 corresponding to a next region to be scanned. The operation of the system is the same as that described in connection with FIG. 3. However, in this embodiment, instead of the graphically displayed scan guidance, a textual based scan guidance is provided.

In this embodiment, the table 92 may be a list of portions of the anatomical region to be scanned or that have been scanned, which may be predetermined, predefined or created by a user, such as based on a desired or required order for scanning the anatomical region. Thus, the table 92 can guide a user as to the order of the scan to identify the next portion of the anatomical region to be scanned. The hierarchical tree structure of the table 92 in various embodiments corresponds to the predefined anatomical folders in memory where acquired images from the current and past exams are stored as described in more detail herein.

Thus, the various embodiments allow a user (e.g., a doctor) to compare past studies with current images to verify treatment progress. A user can use the anatomical legend or customize a table to define a scan sequence, such that a customized study may be provided. Single selection (e.g., one click) operation is also provided to load the correct anatomical reference image, re-calibrate all scan parameters based on the archived image and resume scanning.

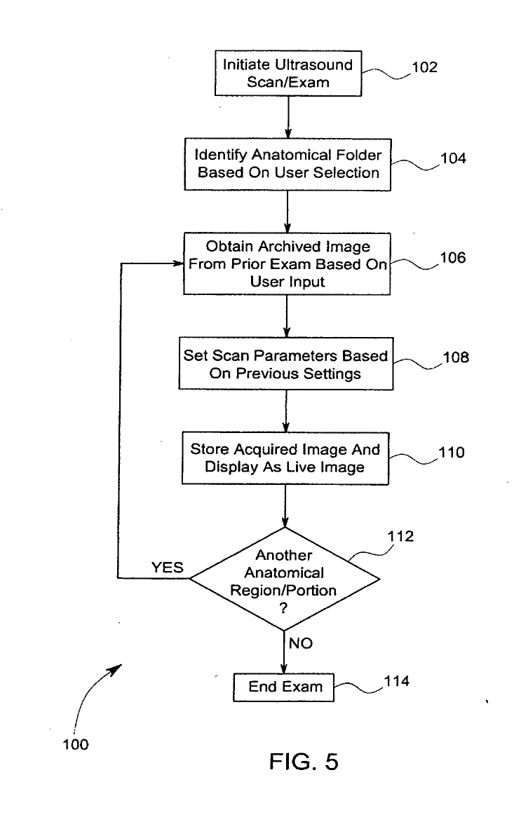

A method 100 as illustrated in FIG. 5 also may be provided in accordance with various embodiments to acquire and store ultrasound data. The method includes initiating an ultrasound scan or exam at 102. For example, a particular ultrasound scan or exam may be selected by a user with a corresponding probe used to acquire image data for that scan or exam. The initiation may set default scan parameters, which subsequently may be adjusted as described herein. Additionally, a display that includes user guidance is provided to allow selection of a particular portion of an anatomical region to be scanned.

A user input selecting the anatomical portion is used to identify an anatomical folder at 104 wherein archived images for the patient being scanned are stored. The anatomical folder may correspond to memory locations where the archived images are stored and organized based on the anatomical regions and portions. Thereafter, an archived image (if any) corresponding to the user input, namely a selected anatomical portion, is obtained at 106. The archived image is an image of the scanned anatomical portion for the patient acquired during an earlier exam, which may be days, weeks, months or years, prior to the current exam.

Based on the previous settings for the ultrasound system when the archived image was acquired, the current scan parameters are set the same. It should be noted that a user may modify or adjust the settings if desired or needed. It also should be noted that this setting of the scan parameters is optionally performed.

Thereafter, an image of the selected anatomical portion is acquired using the ultrasound probe and the image stored in the corresponding anatomical folder at 108. Additionally, the live image is displayed at 108 with the archived image. A determination is then made if another anatomical region or portion is to be scanned, such as based on another user selection in the scan guidance portion of the display. If another anatomical region or portion is selected, then another archived image for that region or portion is obtained at 106 and the method proceeds as described above. If another anatomical region or portion is not selected, then the exam ends at 114.

The various embodiments may also provide additional functionality or processing. For example, a report may be automatically generated with some (or all) of the anatomically aligned images (e.g., images of the same anatomy from multiple scans) grouped together and labeled with the date of the exam. The report can be stored in different formats, and may be stored digitally (e.g., a PDF file, etc.), communicated over a network or printed out.

Thus, the various embodiments provide a system and method for displaying and storing in an anatomical based arrangement ultrasound images from an exam. The selection of the particular anatomical region or portion is performed using, for example, an anatomical image legend highlighting the anatomical region or area corresponding to the displayed archived image, or a customized user-defined table, which includes text, such as a hierarchical tree structure. It should be noted that the user guidance may include any text or graphics to allow a user to select a particular anatomical region or portion of interest and is not limited to the described embodiments.

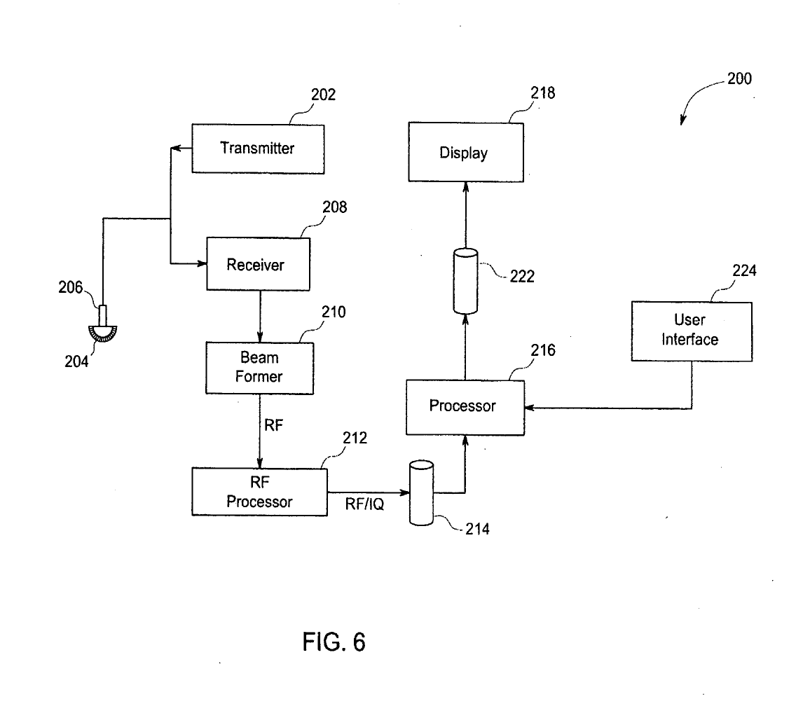

The various embodiments may be implemented in connection with an ultrasound system 200 as illustrated in FIG. 6. The ultrasound system includes a probe 206 for acquiring ultrasound data (e.g., image data) from a patient, which may be used to generate one or more images for display on a display 218. The ultrasound system 200 is capable of electrical or mechanical steering of a soundbeam (such as in 3D space) and is configurable to acquire information corresponding to a plurality of 2D representations or images of a region of interest (ROI) in a subject or patient, which may be defined or adjusted as described in more detail herein. The ultrasound system 200 is configurable to acquire 2D images in one or more planes of orientation.

The ultrasound system 200 includes a transmitter 202 that, under the guidance of a beamformer 210, drives an array of elements 204 (e.g., piezoelectric elements) within the probe 206 to emit pulsed ultrasonic signals into a body. A variety of geometries may be used. The ultrasonic signals are back-scattered from structures in the body, like blood cells or muscular tissue, to produce echoes that return to the elements 204. The echoes are received by a receiver 208. The received echoes are passed through the beamformer 210, which performs receive beamforming and outputs an RF signal. The RF signal then passes through an RF processor 212. Alternatively, the RF processor 212 may include a complex demodulator (not shown) that demodulates the RF signal to form IQ data pairs representative of the echo signals. The RF or IQ signal data may then be routed directly to a memory 214 for storage.

In the above-described embodiment, the beamformer 210 operates as a transmit and receive beamformer. In an alternative embodiment, the probe 206 includes a 2D array with sub-aperture receive beamforming inside the probe. The beamformer 210 may delay, apodize and sum each electrical signal with other electrical signals received from the probe 206. The summed signals represent echoes from the ultrasound beams or lines. The summed signals are output from the beamformer 210 to an RF processor 212. The RF processor 212 may generate different data types, e.g. B-mode, color Doppler (velocity/power/variance), tissue Doppler (velocity), and Doppler energy, for multiple scan planes or different scanning patterns. For example, the RF processor 212 may generate tissue Doppler data for multi-scan planes. The RF processor 212 gathers the information (e.g. I/Q, B-mode, color Doppler, tissue Doppler, and Doppler energy information) related to multiple data slices and stores the data information, which may include time stamp and orientation/rotation information, in the memory 214. It should be noted that in some embodiments a software beamformer (not shown) may be provided in a back end of the ultrasound system 200 such that the ultrasound data is stored in raw form prior to beamforming.

The ultrasound system 200 also includes a processor 216 to process the acquired ultrasound information (e.g., RF signal data or IQ data pairs) and prepare frames of ultrasound information for display on the display 218. The processor 216 is adapted to perform one or more processing operations according to a plurality of selectable ultrasound modalities on the acquired ultrasound data. Acquired ultrasound data may be processed and displayed in real-time during a scanning session as the echo signals are received. Additionally or alternatively, the ultrasound data may be stored temporarily in memory 214 during a scanning session and then processed and displayed in an off-line operation.

The processor 216 is connected to a user interface 224 (which may include a mouse, keyboard, etc.) that may control operation of the processor 116 as explained below in more detail. The display 218 includes one or more monitors that present patient information, including diagnostic ultrasound images to the user for diagnosis and analysis. One or both of memory 214 and memory 222 may store two-dimensional (2D) or three-dimensional (3D) data sets of the ultrasound data, where such 2D and 3D data sets are accessed to present 2D (and/or 3D images) or physiological monitoring data. The acquired image data may be stored in an anatomical based arrangement as described herein. The images may be modified and the display settings of the display 218 also manually adjusted using the user interface 224.

It should be noted that although the various embodiments may be described in connection with an ultrasound system, the methods and systems are not limited to ultrasound imaging or a particular configuration thereof. The various embodiments may be implemented in connection with different types of imaging systems, including, for example, x-ray imaging systems, magnetic resonance imaging (MRI) systems, computed-tomography (CT) imaging systems, positron emission tomography (PET) imaging systems, or combined imaging systems, among others. Further, the various embodiments may be implemented in non-medical imaging systems, for example, non-destructive testing systems such as ultrasound weld testing systems or airport baggage scanning systems.

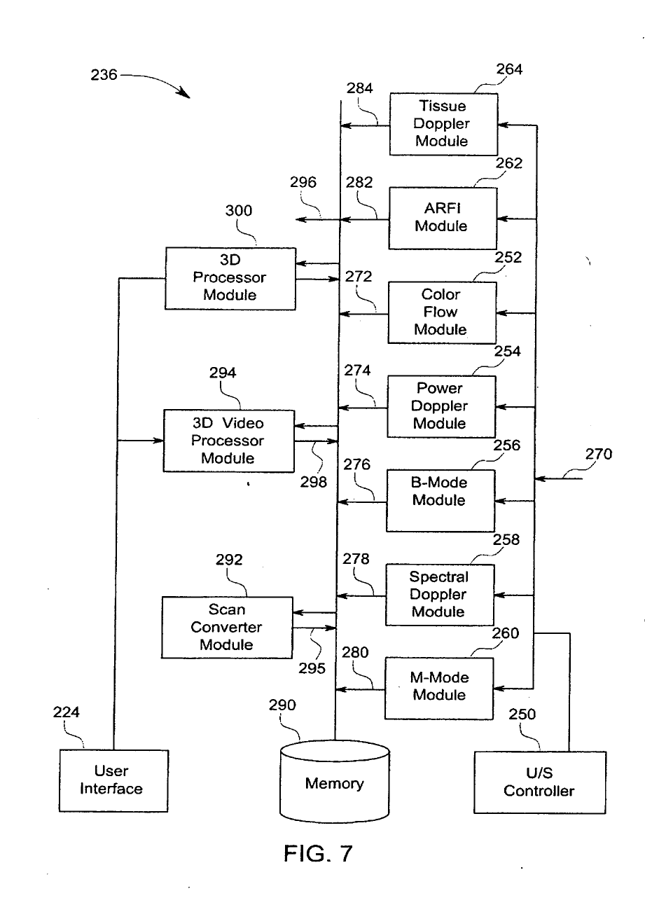

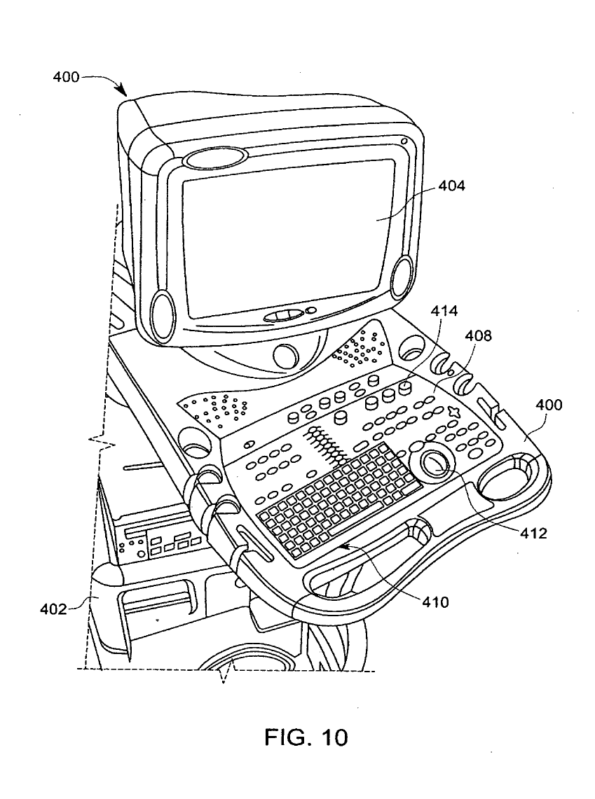

FIG. 7 illustrates an exemplary block diagram of an ultrasound processor module 236, which may be embodied as the processor 216 of FIG. 6 or a portion thereof. The ultrasound processor module 236 is illustrated conceptually as a collection of sub-modules, but may be implemented utilizing any combination of dedicated hardware boards, DSPs, processors, etc. Alternatively, the sub-modules of FIG. 10 may be implemented utilizing an off-the-shelf PC with a single processor or multiple processors, with the functional operations distributed between the processors. As a further option, the sub-modules of FIG. 7 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 7 may be controlled by a local ultrasound controller 250 or by the processor module 236. The sub-modules 252-264 perform mid-processor operations. The ultrasound processor module 236 may receive ultrasound data 270 in one of several forms. In the embodiment of FIG. 6, the received ultrasound data 270 constitutes I,Q data pairs representing the real and imaginary components associated with each data sample. The I,Q data pairs are provided to one or more of a color-flow sub-module 252, a power Doppler sub-module 254, a B-mode sub-module 256, a spectral Doppler sub-module 258 and an M-mode sub-module 260. Optionally, other sub-modules may be included such as an Acoustic Radiation Force Impulse (ARFI) sub-module 262 and a Tissue Doppler (TDE) sub-module 264, among others.

Each of sub-modules 252-264 are configured to process the I,Q data pairs in a corresponding manner to generate color-flow data 272, power Doppler data 274, B-mode data 276, spectral Doppler data 278, M-mode data 280, ARFI data 282, and tissue Doppler data 284, all of which may be stored in a memory 290 (or memory 214 or memory 222 shown in FIG. 5) temporarily before subsequent processing. For example, the B-mode sub-module 256 may generate B-mode data 276 including a plurality of B-mode image planes, such as in a biplane or triplane image acquisition as described in more detail herein.

The data 272-284 may be stored, for example, as sets of vector data values, where each set defines an individual ultrasound image frame. The vector data values are generally organized based on the polar coordinate system.

A scan converter sub-module 292 accesses and obtains from the memory 290 the vector data values associated with an image frame and converts the set of vector data values to Cartesian coordinates to generate an ultrasound image frame 294 formatted for display. The ultrasound image frames 295 generated by the scan converter module 292 may be provided back to the memory 290 for subsequent processing or may be provided to the memory 214 or the memory 222.

Once the scan converter sub-module 292 generates the ultrasound image frames 295 associated with, for example, B-mode image data, and the like, the image frames may be restored in the memory 290 or communicated over a bus 296 to a database (not shown), the memory 214, the memory 222 and/or to other processors.

The scan converted data may be converted into an X,Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the display 218 (shown in FIG. 6), which may include one or more monitors or windows of the display, to display the image frame. The image displayed in the display 218 is produced from image frames of data in which each datum indicates the intensity or brightness of a respective pixel in the display.

Referring again to FIG. 7, a 2D video processor sub-module 294 combines one or more of the frames generated from the different types of ultrasound information. For example, the 2D video processor sub-module 294 may combine a different image frames by mapping one type of data to a grey map and mapping the other type of data to a color map for video display. In the final displayed image, color pixel data may be superimposed on the grey scale pixel data to form a single multi-mode image frame 298 (e.g., functional image) that is again re-stored in the memory 290 or communicated over the bus 296. Successive frames of images may be stored as a cine loop in the memory 290 or memory 222 (shown in FIG. 6). The cine loop represents a first in, first out circular image buffer to capture image data that is displayed to the user. The user may freeze the cine loop by entering a freeze command at the user interface 224. The user interface 224 may include, for example, a keyboard and mouse and all other input controls associated with inputting information into the ultrasound system 200 (shown in FIG. 6).

A 3D processor sub-module 300 is also controlled by the user interface 124 and accesses the memory 290 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

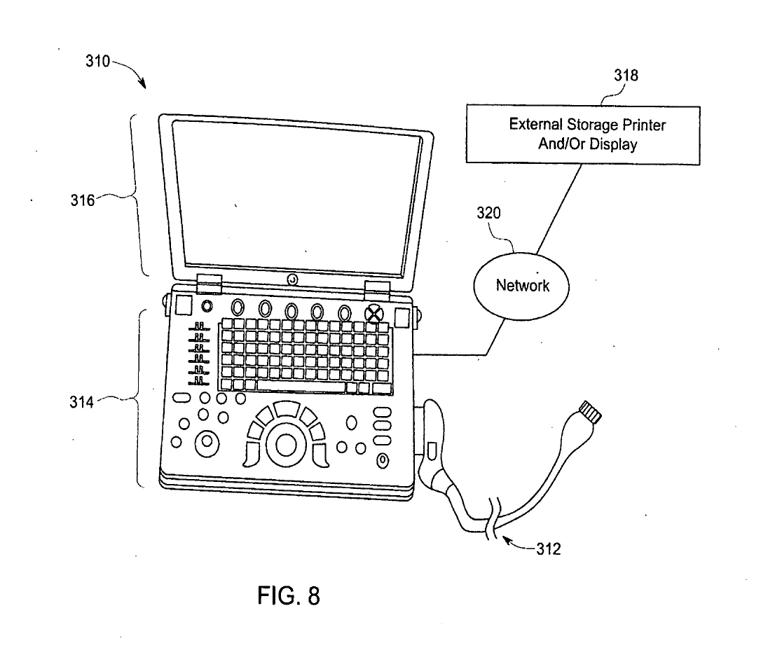

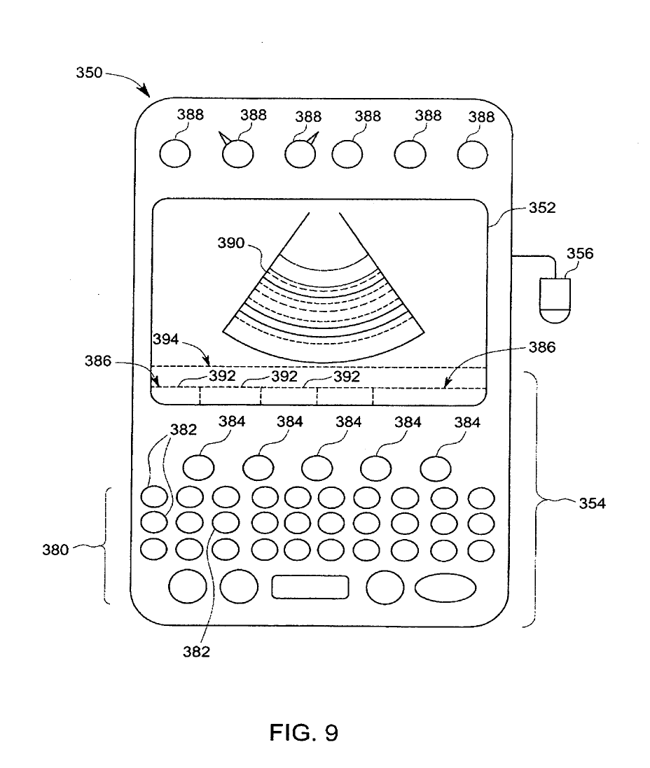

The ultrasound system 200 of FIG. 6 may be embodied in a small-sized system, such as laptop computer or pocket sized system as well as in a larger console-type system. FIGS. 8 and 9 illustrate small-sized systems, while FIG. 10 illustrates a larger system.

FIG. 8 illustrates a 3D-capable miniaturized ultrasound system 310 having a probe 312 that may be configured to acquire 3D ultrasonic data or multi-plane ultrasonic data. For example, the probe 312 may have a 2D array of elements 204 as discussed previously with respect to the probe 206 of FIG. 6. A user interface 314 (that may also include an integrated display 316) is provided to receive commands from an operator. As used herein, “miniaturized” means that the ultrasound system 310 is a handheld or hand-carried device or is configured to be carried in a person's hand, pocket, briefcase-sized case, or backpack. For example, the ultrasound system 310 may be a hand-carried device having a size of a typical laptop computer. The ultrasound system 330 is easily portable by the operator. The integrated display 316 (e.g., an internal display) is configured to display, for example, one or more medical images.

The ultrasonic data may be sent to an external device 318 via a wired or wireless network 320 (or direct connection, for example, via a serial or parallel cable or USB port). In some embodiments, the external device 318 may be a computer or a workstation having a display, or the DVR of the various embodiments. Alternatively, the external device 318 may be a separate external display or a printer capable of receiving image data from the hand carried ultrasound system 310 and of displaying or printing images that may have greater resolution than the integrated display 316.

FIG. 9 illustrates a hand carried or pocket-sized ultrasound imaging system 350 wherein the display 352 and user interface 354 form a single unit. By way of example, the pocket-sized ultrasound imaging system 350 may be a pocket-sized or hand-sized ultrasound system approximately 2 inches wide, approximately 4 inches in length, and approximately 0.5 inches in depth and weighs less than 3 ounces. The pocket-sized ultrasound imaging system 350 generally includes the display 352, user interface 354, which may or may not include a keyboard-type interface and an input/output (I/O) port for connection to a scanning device, for example, an ultrasound probe 356. The display 352 may be, for example, a 320×320 pixel color LCD display (on which a medical image 190 may be displayed). A typewriter-like keyboard 380 of buttons 382 may optionally be included in the user interface 354.

Multi-function controls 384 may each be assigned functions in accordance with the mode of system operation (e.g., displaying different views). Therefore, each of the multi-function controls 384 may be configured to provide a plurality of different actions. Label display areas 386 associated with the multi-function controls 384 may be included as necessary on the display 352. The system 350 may also have additional keys and/or controls 388 for special purpose functions, which may include, but are not limited to “freeze,” “depth control,” “gain control,” “color-mode,” “print,” and “store.”

One or more of the label display areas 386 may include labels 392 to indicate the view being displayed or allow a user to select a different view of the imaged object to display. The selection of different views also may be provided through the associated multi-function control 384. The display 352 may also have a textual display area 394 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 350 and the miniaturized ultrasound system 300 may provide the same scanning and processing functionality as the system 200 (shown in FIG. 6).

FIG. 10 illustrates an ultrasound imaging system 400 provided on a movable base 402. The portable ultrasound imaging system 400 may also be referred to as a cart-based system. A display 404 and user interface 406 are provided and it should be understood that the display 404 may be separate or separable from the user interface 406. The user interface 406 may optionally be a touchscreen, allowing the operator to select options by touching displayed graphics, icons, and the like.

The user interface 406 also includes control buttons 408 that may be used to control the portable ultrasound imaging system 400 as desired or needed, and/or as typically provided. The user interface 406 provides multiple interface options that the user may physically manipulate to interact with ultrasound data and other data that may be displayed, as well as to input information and set and change scanning parameters and viewing angles, etc. For example, a keyboard 410, trackball 412 and/or multi-function controls 414 may be provided.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, solid state disk drive (e.g., flash drive of flash RAM) and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, the embodiments are by no means limiting and are exemplary embodiments. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for storing ultrasound data, the method comprising:
displaying selectable anatomical identification guidance information having a plurality of identifiers corresponding to a plurality of anatomical portions of an anatomical region;
receiving a user input selecting one of the plurality of identifiers; and
storing a subsequently acquired image and associating the stored image with the anatomical potion of the anatomical region corresponding to the selected identifier.

2. The method of claim 1, further comprising displaying at least one archived image corresponding to a selected anatomical portion of the anatomical region.

3. The method of claim 2, further comprising setting scan parameters for an ultrasound probe to settings based on settings used to acquire the at least one archived image.

4. The method of claim 1, wherein the storing comprises storing images in predefined anatomical folders corresponding to the anatomical portion and region.

5. The method of claim 1, further comprising displaying a plurality of selectable thumbnail archived images from previous exams.

6. The method of claim 1, further comprising storing with the subsequently acquired image, scan parameters used to acquire the image.

7. The method of claim 1, wherein the selectable anatomical identification guidance information comprises an anatomical image legend and further comprising highlighting a selected portion.

8. The method of claim 7, wherein the anatomical image legend defines a shape corresponding to the anatomical region to be imaged.

9. The method of claim 7, further comprising displaying text corresponding to a portion of the anatomical image legend selected.

10. The method of claim 1, wherein the selectable anatomical identification guidance information comprises a text table and further comprising highlighting a selected table entry.

11. The method of claim 10, wherein the text table is user defined.

12. The method of claim 10, wherein the text table comprises a hierarchical arrangement of the anatomical portions and regions.

13. The method of claim 1, wherein the storing comprises archiving stored image data one of locally on an ultrasound system or remotely from the ultrasound system, the remote storage communicatively coupled to the ultrasound system via one of a wired, wireless or internet network.

14. The method of claim 1, further comprising storing, in connection with stored images, a definition of the image processing algorithm used to process the stored images.

15. The method of claim 1, further comprising displaying at least one live image and at least one stored image of an identified anatomical portion based on the selected identifier, and applying post-processing parameters to the displayed live and stored images.

16. The method of claim 1, further comprising automatically generating a report having a plurality of images grouped together for an identified anatomical portion based on the selected identifier.

17. An ultrasound display comprising:
a first section displaying a currently acquired ultrasound image of an anatomical portion of a patient;
a second section displaying an archived ultrasound image of the anatomical portion of the patient; and
a third section displaying selectable anatomical identification guidance information having a highlighted identifier corresponding to the anatomical portion.

18. The ultrasound display of claim 17, wherein the currently acquired image and the archived ultrasound image are displayed side by side.

19. The ultrasound display of claim 17, wherein the selectable anatomical identification guidance information comprises an anatomical image legend and the highlighted identifier corresponds to a portion of the anatomical image legend selected.

20. The ultrasound display of claim 19, wherein the anatomical image legend is a displayed shape corresponding to the anatomical region to be imaged.

21. The ultrasound display of claim 20, further comprising text displayed corresponding to a portion of the anatomical image legend selected.

22. The ultrasound display of claim 17, wherein the selectable anatomical identification guidance information comprises a text table and the highlighted identifier corresponds to a selected table entry.

23. An ultrasound system comprising:
a probe configured to acquire ultrasound image data;
a memory storing the acquired ultrasound image data using a predefined anatomical identification arrangement;
a processor configured to obtain archived ultrasound image data; and
a display configured to display an image based on the acquired image data and an image based on the archived image data stored in the memory, and further displaying selectable anatomical identification guidance information having a highlighted identifier corresponding to an anatomical portion for the displayed images.

24. The ultrasound system of claim 23, wherein the selectable anatomical identification guidance information comprises one of (i) an anatomical image legend and the highlighted identifier corresponds to a portion of the anatomical image legend selected or (ii) a text table and the highlighted identifier corresponds to a selected table entry.
